(19) United States
(12) Patent Application Publication
(10) Pub. No.: US 2025/0123277 A1
(43) Pub. Date: Apr. 17, 2025

(54) ENVIRONMENTAL BIOSPECIMEN RECOVERY AFTER IN-DROPLET GEL ENCAPSULATION

(71) Applicant: The Texas A&M University System, College Station, TX (US)

(72) Inventors: Arum Han, College Station, TX (US); Rohit Kunal Gupte, College Station, TX (US); Jing Dai, College Station, TX (US); Paul J. de Figueiredo, College Station, TX (US); Erin J. Van Schaik, Bryan, TX (US)

(73) Assignee: The Texas A&M University System, College Station, TX (US)

(21) Appl. No.: 18/721,703

(22) PCT Filed: Dec. 23, 2022
(86) PCT No.: PCT/US2022/053979

§ 371 Date: Jun. 19, 2024

Related U.S. Application Data
(60) Provisional application No. 63/294,298, filed on Dec. 28, 2021.

(51) Int. Cl. G01N 33/569; C12N 1/20; C12N 11/04

(52) U.S. Cl. CPC G01N 33/56911; C12N 1/20; C12N 11/04; C12N 2509/00; C12N 2531/00

(57) ABSTRACT

In an embodiment. the present disclosure pertains to environmental biospecimen recovery after in-droplet gel encapsulation (eBRIDGE) platforms for co-culturing multiple microorganisms in gel microspheres and then transferring single-cell-derived clonal populations from within the gel microspheres into separate water-in-oil emulsion droplets for further processing and analysis. In some embodiments. the gel-encapsulated bacteria are released by lysing the gel matrix using an enzyme. The methods of the present disclosure provide a single workflow that goes from environmental microbial harvesting and amplification to functional interrogation of their characteristics.

### CROSS-REFERENCE TO RELATED APPLICATIONS

This patent application claims priority from, and incorporates by reference the entire disclosure of, U.S. Provisional Application 63/294,298 filed on Dec. 28, 2021.

### STATEMENT REGARDING FEDERALLY SPONSORED RESEARCH

This invention was made with government support under W911NF1920013 awarded by the Defense Advanced Research Projects Agency. The government has certain rights in the invention.

### TECHNICAL FIELD

The present disclosure relates generally to biospecimen recovery and more particularly, but not by way of limitation, to environmental biospecimen recovery after in-droplet gel encapsulation (eBRIDGE).

### BACKGROUND

This section provides background information to facilitate a better understanding of the various aspects of the disclosure. It should be understood that the statements in this section of this document are to be read in this light, and not as admissions of prior art.

The emergence of new culture strategies has recently enabled the isolation and characterization of rare or unculturable species of bacteria derived from the environment. However, currently available techniques lead to time-consuming and labor-intensive workflows. No existing technology is capable of harvesting grown single-cell derived colonies of microorganisms or mammalian cells from multi-strain co-cultures, followed by preparing them ready for high-throughput phenotyping/functional assays especially those utilizing droplet microfluidics, in an automated and high-throughput manner.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a method to encapsulate at least one of individual microbial cells from a single-or multi-organism suspension, individual mammalian cells from a genetically heterogenous population, or individual mammalian cells from a genetically identical population into gel microspheres. In general, the method includes: (1) encapsulating single-cells into the gel microspheres using a gel material; (2) cultivating the gel microspheres to produce viable single-cell-derived colonies of encapsulated organisms; (3) transitioning each single-cell derived colony into separate water-in-oil emulsion droplets; and (4) releasing cellular cargo from the gel microspheres using enzyme-based degradation of the gel material used for encapsulation into the water-in-oil emulsion droplets.

In an additional embodiment, the present disclosure pertains to a method to encapsulate at least one of individual microbial cells from a single-or multi-organism suspension, individual mammalian cells from a genetically heterogenous population, or individual mammalian cells from a genetically identical population into gel microspheres. In general, the method includes: (1) encapsulating single-cells in gel microdroplets using a gel material; (2) cultivating the gel microspheres to produce viable single-cell derived colonies of encapsulated microorganisms; (3) transitioning each single-cell derived colony into separate water-in-oil emulsion droplets; and (4) releasing the cellular cargo from the gel microspheres using a temperature-dependent dissolution/degradation of the gel material used for encapsulation.

In a further embodiment, the present disclosure pertains to a method to create co-cultures of various distinct microbes within a diverse community, or genetically distinct mammalian cells within a population of genetically distinct mammalian cells according to the methods disclosed herein.

In another embodiment, the present disclosure pertains to a method to pair microspheres made of different gel materials with cells from a single-organism microbial culture or population of genetically identical mammalian cells to assay a target organism's ability to interact with, adhere to, consume or degrade various gel materials according to the methods disclosed herein.

In a further embodiment, the present disclosure pertains to a method to pair single cells from multi-organism mixtures, or a population of genetically identical cells, microbial or mammalian, with microspheres made of a specific gel material to identify and recover specific species or strains based on their ability to interact with, adhere to, consume or degrade the gel material using a method according to the methods disclosed herein.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1 illustrates an example environmental biospecimen recovery after in-droplet gel encapsulation (eBRIDGE) workflow according to an aspect of the present disclosure.

FIGS. 2A-2F illustrate an experimental workflow of eBRIDGE (Implementation #1) according to an aspect of the present disclosure. Green fluorescent protein (GFP)-Escherichia coli (E. coli) and red fluorescent protein (RFP)-Salmonella enterica (S. enterica) are illustrated here as examples of two mixed strains in a community. FIG. 2A illustrates a gel droplet generation chip and gel droplet re-encapsulation chip for the workflow of eBRIDGE (Implementation #1). FIG. 2B shows single-cell encapsulation of GFP-E. coli and RFP-S. enterica in agarose gel microdroplets using a microfluidic gel microsphere generator. FIG. 2C shows agarose droplets containing single bacterial cells are washed to remove the carrier oil, re-suspended in aqueous culture media, and then co-cultured as communities. Diffusion-mediated inter-cell communications are maintained and the exchange of various metabolites and/or outer membrane vesicles (OMVs) is illustrated here. This allows the encapsulated single cells to be expanded, resulting in single-cell-derived colonies of bacteria within the gel microspheres. FIG. 2D shows the agarose droplets are washed and packed tightly into a single line using the elliptical “droplet packing” microchannel of the microfluidic chip. Tight packing is needed to ensure highly efficient single-gel-droplet encapsulation into water-in-oil emulsion droplets. FIG. 2E shows the “packed” droplets are re-flown with culture media containing agarase enzyme and encapsulated into water-in-oil droplets at single-droplet resolution. FIG. 2F shows the agarase enzyme degrades the agarose gel microsphere, releasing the gel-encapsulated cells into the water-in-oil emulsion droplet for further culture and analysis. Cells in each droplet are clonal populations of the single cells initially encapsulated into each gel microsphere. These cells are now ready for further downstream droplet microfluidics-based phenotypic analysis.

FIGS. 3A-3F illustrate an experimental workflow of eBRIDGE (Implementation #2) according to another aspect of the present disclosure. GFP-E. coli and RFP-S. enterica are illustrated here as examples of two mixed strains in a community. FIG. 3A illustrates a gel droplet generation chip and gel droplet re-encapsulation chip for the workflow of eBRIDGE (Implementation #2). FIG. 3B shows single-cell encapsulation of GFP-E. coli and RFP-S. enterica in temperature-sensitive gel microdroplets using a microfluidic gel microsphere generator. FIG. 3C shows gel microspheres containing single bacterial cells are washed to remove the carrier oil, re-suspended in aqueous culture media, and then co-cultured as communities. Diffusion-mediated inter-cell communications are maintained and the exchange of various metabolites and/or OMVs is illustrated here. This allows the encapsulated single cells to be expanded, resulting in single-cell-derived colonies of bacteria within the gel microspheres. FIG. 3D shows the gel microspheres are washed and packed tightly into a single line using the elliptical “droplet packing” microchannel of the microfluidic chip. Tight packing is needed to ensure highly efficient single-gel-droplet encapsulation into water-in-oil droplets. FIG. 3E shows the “packed” droplets are re-flown with culture media and encapsulated into water-in-oil emulsion droplets at single-droplet resolution. FIG. 3F shows the re-encapsulated gel microspheres can be dissolved by either incubating the collected droplets at the desired temperature off chip, or by infusing hot or cold water through a temperature regulation microchannel fabricated below the re-encapsulation region of the chip. A temperature-triggered dissolution of the gel matrix can be initiated by this temperature change, releasing the gel-encapsulated cells into the water-in-oil emulsion droplet for further culture and analysis. Cells in each droplet are clonal populations of the single cells initially encapsulated into each gel microsphere. These cells are now ready for further downstream droplet microfluidics-based phenotypic analysis.

FIG. 4 illustrates an example of an eBRIDGE platform (Implementation #3) according to a further aspect of the present disclosure.

FIG. 5 illustrates an example of an eBRIDGE platform (Implementation #4) according to an additional aspect of the present disclosure.

FIGS. 6A-6B illustrate a schematic of the gel microsphere to water-in-oil emulsion droplet transfer device. FIG. 6A illustrates the schematic of the gel droplet to water-in-oil emulsion droplet transfer device. FIG. 6B illustrates a zoomed in view of a junction point of the gel droplets, oil, and culture media. The height of the gel microsphere re-encapsulation part of this device (top) is adjusted to be slightly smaller than the gel microspheres themselves. This ensures efficient packing of the gel microspheres. The imaging chamber is 100 μm in height. This ensures that the water-in-oil emulsion droplets are not squeezed during imaging.

FIGS. 7A-7C illustrate characterization of the purified agarase GH86 activities in degrading agarose. FIG. 7A shows the standard curve of dinitro salicylate (DNS) activity assay. The DNS reagents were incubated with galactose standards at different concentrations (1 μM to 250 nM), and absorbance measured at 540 nm. FIG. 7B shows relative activity of the purified agarase enzyme. Various concentrations of agarase were incubated with 1 mg/mL agarose and the relative activity was measured using the DNS assay. Error bars are standard deviation. N=3. FIG. 7C shows analysis of images showing the degradation of gel microdroplets made of 1% and 2% agarose using the agarase enzyme (2 mg/ml agarase was found to work best at a temperature of 37°° C.). The number of observable agarose droplets reduced significantly in the presence of the agarase enzyme. Fewer droplets are observed at 37° C. than at room temperature (RT).

FIG. 8 illustrates analysis of the water-in-oil droplets content. Fluorescence analysis of ˜1800 droplets shows very low correlation between the GFP and RFP intensities of the water-in-oil droplets. A scatter plot of the GFP and RFP intensities shows an L-shaped distribution of twoindependent variables, suggesting a good degree of separation between the GFP and RFP microbes.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Over 99% of the extant microbial biodiversity has yet to be characterized. This phenomenon is known as the great plate count anomaly, and is due to the inability to grow most environmental microorganisms in a laboratory setting. A poor imitation of their native microenvironment and disruption of essential inter-cell communication pathways that these microbes are dependent on, leads to most rare and delicate species of microorganisms unable to grow in traditional cultivation methods. The emergence of new cultivation strategies has recently enabled the isolation and characterization of rare/unculturable species of microorganisms derived from the environment. Some of these strategies have the advantages of being able to physically isolate environmental microorganisms and cultivate them in their native microenvironment, while others have demonstrated the survival of previously unculturable microorganisms in a laboratory environment through co-cultivation. All of these techniques have some things in common: (1) they all rely on physically isolating single microbial cells while maintaining diffusion-mediated inter-cellular communication during culture; (2) the resulting bacterial colonies need to be harvested manually for further analyses; and (3) the harvested colonies need to be characterized using conventional microbiology techniques that are manual and labor-intensive.

All these characteristics lead to time-consuming and labor-intensive workflows. No existing technology is capable of harvesting clonal populations of grown colonies of microorganisms from a multi-strain co-culture in an automated and high-throughput manner, followed by preparing them ready for high-throughput phenotyping assays.

Disclosed herein is a new technology named environmental biospecimen recovery after in-droplet gel encapsulation (eBRIDGE), a platform for co-culturing multiple microorganisms in gel microspheres and then seamlessly transferring the single-cell-derived clonal populations from within the microspheres into water-in-oil emulsion droplets for further processing and analyses. The gel-encapsulated microbes are released into the water-in-oil emulsion droplets by degrading the gel matrix. In one such example, agarose gel microspheres can be used to encapsulate the cells, where the gel matrix can be degraded by an agarase enzyme. Taken together, this disclosure presents a single workflowthat goes from environmental microbial harvest and amplification to functional interrogation of their characteristics in a high-throughput manner.

Applications that seek to benefit from the diverse activities and functions of

environmental microbes benefit from the systems and methods disclosed herein. For example, any application that seeks to screen the diverse activities of environmental microbes through a phenotypic assay can benefit from eBRIDGE, which provides, for the first time, high-throughput workflow throughout the entire discovery process.

For example, eBRIDGE can link microbe-encapsulated gel microspheres co-culture

strategies to high-throughput droplet microfluidics-based phenotyping assays so that the entire workflow is high throughput. Co-cultured microbial strains in gel microspheres cannot be easily rendered into high-throughput phenotyping assays, where the cultured cells are typically dispensed into an agar plate or individual wells of a multi-well plate, followed by picking those colonies for further analysis. This recovery process is usually performed manually, leading to a very labor intensive and low throughput workflow. Even when using an automated colony picker and/or liquid handling robot, the throughput is still limited or the cost is high. Importantly, once the cells are moved to an off-chip cultivation environment (e.g., well plate, agar plate, and the like), they cannot benefit from the extremely high-throughput nature of droplet microfluidics. The technology disclosed herein automates the process of recovering colonies of genetically identical microbes from a multi-strain co-culture, making characterization or further experimentation on the recovered colonies straightforward, high throughput, and low in cost.

Encapsulation of environmental microbes, at single-cell resolution, into gel microspheres, co-cultivating them, followed by washing (to remove all microbes that are not encapsulated within a gel microsphere or grown out of the gel microsphere), re-encapsulation of the gel microspheres into a water-in-oil emulsion droplets where a single gel microsphere is encapsulated into a single water-in-oil emulsion droplet (1:1 transfer), addition of gel-dissolving molecules (e.g., agarase enzyme in the case of agarose gel microspheres) that degrades the gel so that microbes within the gel microspheres can be released into aqueous phase of the water-in-oil emulsion droplet. The final outcome is clonally expanded population of the initial environmental microbes placed within a water-in-oil emulsion droplet and ready to be screened in high-throughput droplet microfluidics-based phenotyping assays.

Current technologies allow users to capture, isolate, and co-culture environmental bacteria in order to promote the survival and proliferation of previously unculturable strains. However, the number of microbial capture chambers are limited (maximum several hundred to thousands), and then transferring the cultured microbes from each chamber for further analysis require manual transfer one by one. Gel microsphere encapsulation of environmental microbes followed by cultivation is another commonly used technique to culture the unculturable, which can then be readily transferred to a well plate or agar plate for further analysis. However, conducting high-throughput assays on the transferred cells are difficult. Thus, no technology currently exists that can quickly and efficiently recover the cultured bacteria for further phenotypic characterization and experimentation.

### Working Examples

Reference will now be made to more specific embodiments of the present disclosure and data that provides support for such embodiments. However, it should be noted that the disclosure below is for illustrative purposes only and is not intended to limit the scope of the claimed subject matter in any way.

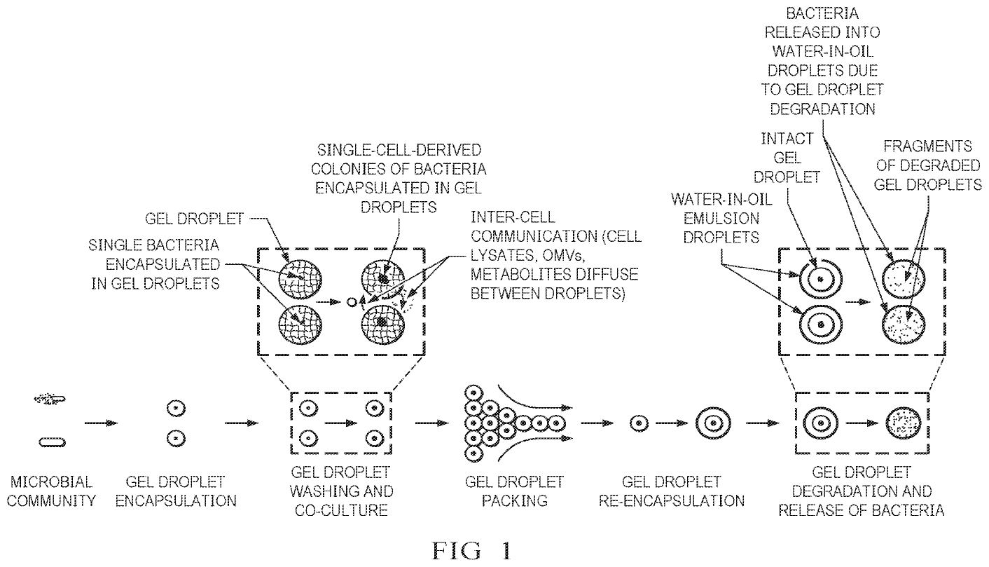

FIG. 1 illustrates an eBRIDGE workflow according to an aspect of the present disclosure. First, the eBRIDGE process begins with a microbial community harvested from the environment, or a prepared mammalian cell culture (herein referred to as a “sample”). Then, single cells from the sample are encapsulated in separate gel droplets. In some embodiments, any gel material can be used including, but not limited to, agarose, polyethylene glycol (PEG), gelatin methacryloyl, MEBIOL®, alginate, and combinations of the same and like. Next, the gel is allowed to set in various ways depending on the gel chemistry, then the gel droplets are washed to remove the carrier oil, re-suspended in aqueous culture media (e.g., Reasoner's 2A agar (R2A), lysogeny broth (LB), trypticase soy broth (TSB), M3™, and combinations of the same and like) and then co-cultured as communities. Diffusion mediated inter cell communication pathways, which can be important for the survival of some delicate, rare, and/or previously unculturable species of bacteria, are maintained and the exchange of various metabolites and/or outer membrane vesicles (OMVs) is illustrated here. In some embodiments, this allows the encapsulated single cells to be expanded, resulting in single-cell-derived colonies of bacteria within the gel droplets. Afterwards, the gel droplets are then rinsed, compacted by centrifugation, and infused on to a microfluidic chip that packs them tightly into a single line using a “droplet packing” channel. In some instances, tight packing is needed to ensure highly efficient single-gel-droplet encapsulation into water-in-oil droplets. The “packed” droplets are re-encapsulated with culture media into water-in-oil droplets at single-droplet resolution (e.g., one gel droplet re-encapsulated into one water-in-oil droplet). The re-encapsulated gel droplets can then be dissolved either by initiating a temperature-triggered dissolution of the gel droplets (i.e., re-encapsulated droplets can be incubated at the desired temperature off-chip, or, hot or cold water can be flown through a temperature regulation channel fabricated below the re-encapsulation region of the chip) or by including a chemical degrading agent (e.g., agarose GH86, or combinations of the same and like, depending on the gel chemistry) in the re-encapsulated droplets. The released cells are now ready for further downstream droplet microfluidics-based phenotypic analysis.

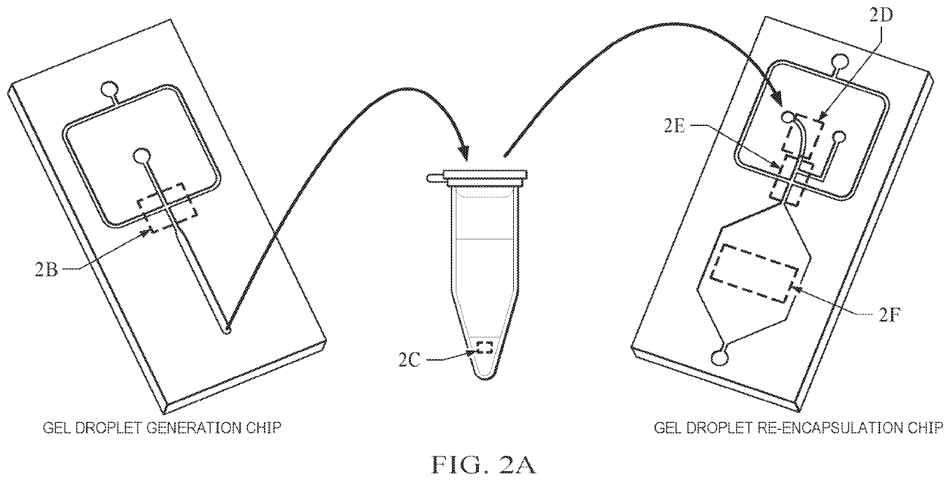

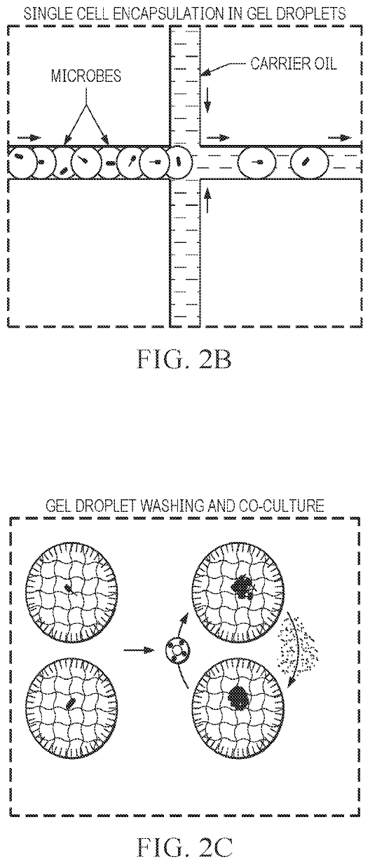

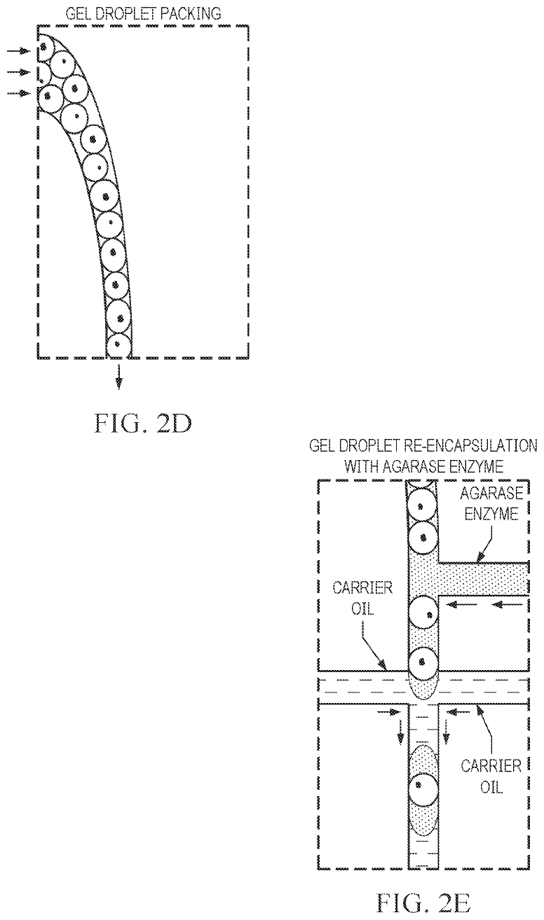

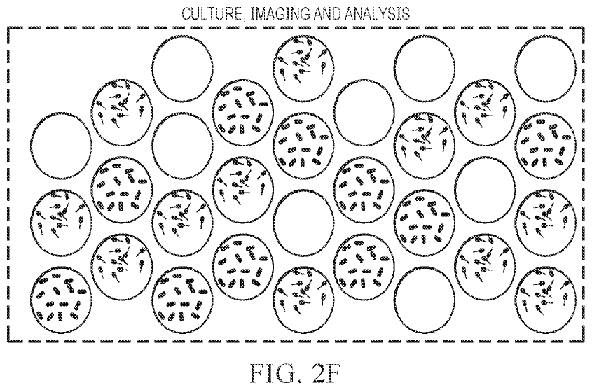

Implementation #1 of the eBRIDGE workflow (FIGS. 2A-2F). Green fluorescent protein (GFP)-Escherichia coli (E. coli) and red fluorescent protein (RFP)-Salmonella enterica (S. enterica) are illustrated here as examples of two strains in a community. FIG. 2A illustrates a gel droplet generation chip and gel droplet re-encapsulation chip for the workflow of eBRIDGE (Implementation #1). FIG. 2B shows single-cell encapsulation of GFP-E. coli and RFP-S. enterica in agarose gel microdroplets using a microfluidic gel droplet generator. FIG. 2C shows agarose droplets containing single bacterial cells are washed to remove the carrier oil, re-suspended in aqueous culture media, and then co-cultured as communities. In some embodiments, diffusion mediated inter cell communication pathways are maintained, and the exchange of various metabolites and/or OMVs is illustrated here. In some embodiments, this allows the encapsulated single cells to be expanded, resulting in single-cell-derived colonies of bacteria within the gel droplets. FIG. 2D shows the agarose droplets are washed and packed tightly into a single line using the elliptical “droplet packing” channel of the microfluidic chip. In some embodiments, tight packing is needed to ensure highly efficient single-gel-droplet encapsulation into water-in-oil droplets. FIG. 2E shows the “packed” droplets are re-flown with culture media containing 20% (v/v) agarase enzyme and encapsulated into water-in-oil droplets at single-droplet resolution. FIG. 2F shows the agarase enzyme degrades the agarose gel droplet, releasing the gel-encapsulated cells into the water-in-oil emulsion droplet for further culture and analysis. In some embodiments, cells in each droplet are clonal populations of the single cells initially encapsulated into each gel droplet. In some embodiments, these cells are now ready for further downstream droplet microfluidics-based phenotypic analysis (gel droplet degradation to release cells, cell culture, and analysis).

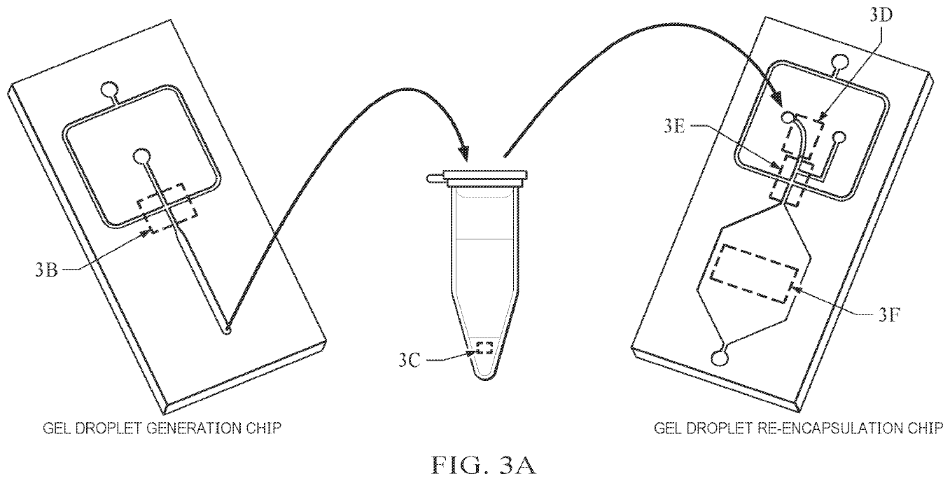

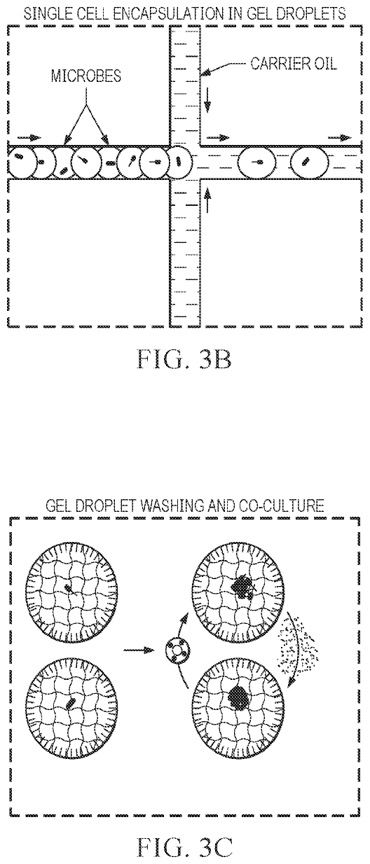

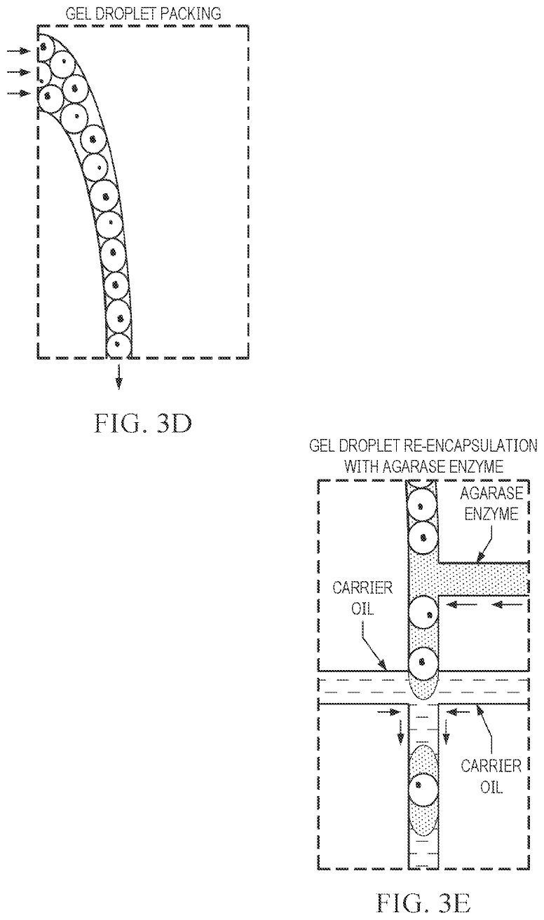

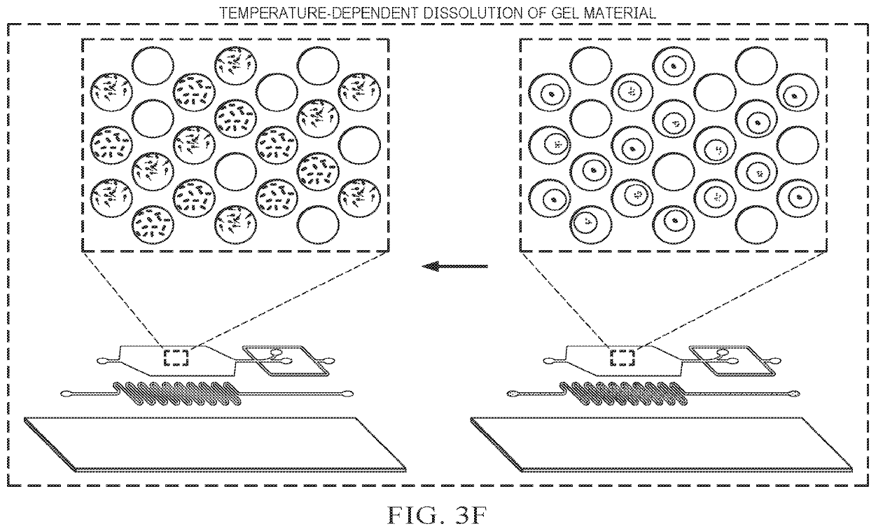

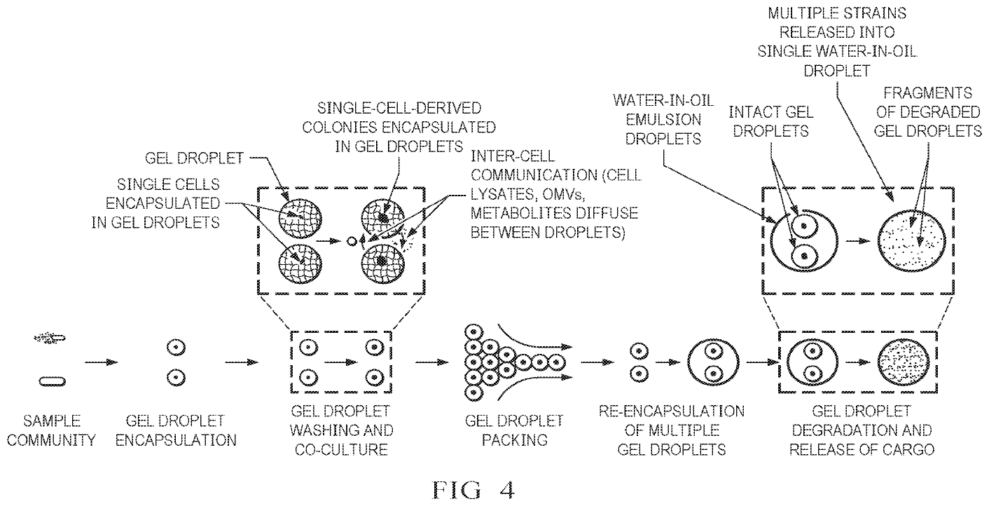

Implementation #2 of the eBRIDGE workflow (FIGS. 3A-3F). GFP-E. coli and RFP-S. enterica are illustrated here as examples of two strains in a community. FIG. 3A illustrates a gel droplet generation chip and gel droplet re-encapsulation chip for the workflow of eBRIDGE (Implementation #2). FIG. 3B shows single-cell encapsulation of GFP-E. coli and RFP-S. enterica in agarose gel microdroplets using a microfluidic gel droplet generator. FIG. 3C shows agarose droplets containing single bacterial cells are washed to remove the carrier oil, re-suspended in aqueous culture media, and then co-cultured as communities. In some embodiments, diffusion mediated inter cell communication pathways are maintained, and the exchange of various metabolites and/or OMVs is illustrated here. In some embodiments, this allows the encapsulated single cells to be expanded, resulting in single-cell-derived colonies of bacteria within the gel droplets. FIG. 3D shows the agarose droplets are washed and packed tightly into a single line using the elliptical “droplet packing” channel of the microfluidic chip. In some instances, tight packing is needed to ensure highly efficient single-gel-droplet encapsulation into water-in-oil droplets. FIG. 3E shows the “packed” droplets are re-flown with culture media and encapsulated into water-in-oil droplets at single-droplet resolution. FIG. 3F shows the re-encapsulated gel droplets can be dissolved by either incubating the collected droplets at the desired temperature off chip, or by infusing hot or cold water through a temperature regulation channel fabricated below the re-encapsulation region chip. In some embodiments, a temperature-triggered dissolution of the gel matrix can be initiated this way, releasing the gel-encapsulated cells into the water-in-oil emulsion droplet for further culture and analysis. In some embodiments, cells in each droplet are clonal populations of the single cells initially encapsulated into each gel droplet. In some embodiments, these cells are now ready for further downstream droplet microfluidics-based phenotypic analysis (gel droplet degradation to release cells, cell culture, and analysis).

With respect to the above-mentioned implementations, the eBRIDGE technology starts by encapsulating microbes in gel microspheres (ideally in single-cell resolution), followed by co-cultivating the gel microspheres (FIG. 2B and FIG. 2C, and FIG. 3B and FIG. 3C). Next, the gel microspheres are encapsulated into water-in-oil emulsion droplets in one-to-one scheme (one gel microsphere into one water-in-oil emulsion droplet) by: packing the gel microspheres to be delivered to the water-in-oil emulsion droplet generator in single file (FIG. 2D and FIG. 3D), and introducing gel-degrading reagents to be co-encapsulated with the gel microspheres (FIG. 2E and FIG. 3E). The incubation of these droplets containing gel microspheres and gel-degradation reagents facilitates the degradation of the gel matrix, thus releasing the bacterial cargo from the gel microspheres into the water-in-oil emulsion for downstream phenotypic analysis (FIG. 2F and FIG. 3F).

Implementation #3 of the eBRIDGE platform (FIG. 4). In some embodiments, single cells from a multi-strain sample (e.g., mammalian, bacterial or other microbial cells) are encapsulated in a gel microsphere similar to FIG. 1. In some embodiments, the gel droplets are washed, resuspended in an aqueous culture medium, and co-cultured to develop single-cell-derived colonies. Then the gel droplets are compacted and packed similar to FIG. 1. In some embodiments, during re-encapsulation, the parameters (e.g., flow rates) of the re-encapsulation device can be modified to ensure that multiple (e.g., two or more) gel droplets are encapsulated in a single water-in-oil droplet. In some instances, the gel is then dissolved/degraded similar to FIGS. 2A-2F or FIGS. 3A-3F depending on the chemistry of the gel. In some embodiments, this process results in distinct pairs of strains from the original sample being co-cultured together in a water-in-oil droplet. In some embodiments, interactions between these various strain combinations can be studied using subsequent interrogation assays.

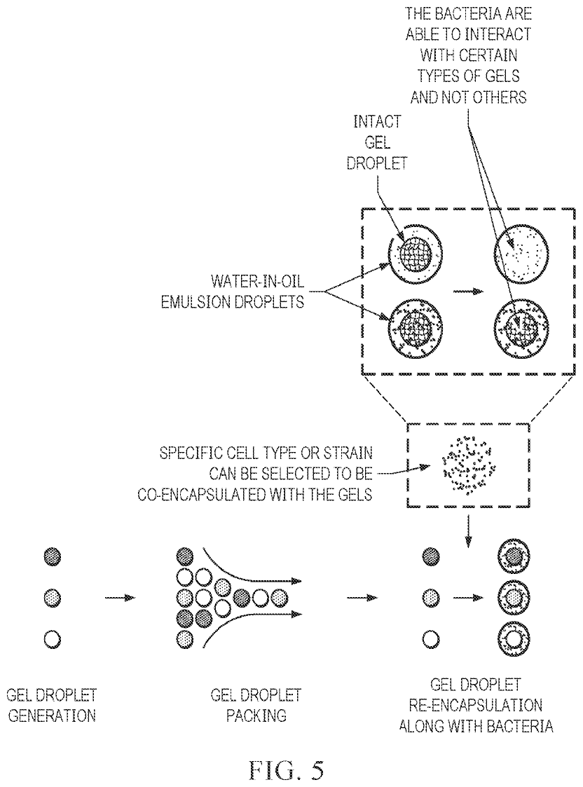

Implementation #4 of the eBRIDGE workflow (FIG. 5). In some embodiments, various gel materials can be emulsified into monodispersed (i.e., of the same size) microspheres using conventional microfluidic droplet generators. In some embodiments, the gels are allowed to set and then mixed together. In some embodiments, the mixture is then compacted and packed similar to FIG. 1. In some embodiments, the packed droplets can then be re-encapsulated along with a specific cell type or bacterial strain. In some embodiments, this results in distinct gel-cell combinations inside the resulting water-in-oil droplets. In some instances, the selected cell types could be capable of interacting with certain gel types, but not others. In some embodiments, these interactions (e.g., degradation, consumption, and combinations of the same and like) can be studied using subsequent interrogation assays.

The systems and methods disclosed herein can be used in conjunction with any droplet microfluidics platform to perform phenotypic interrogation assays on the recovered populations of the microbes. It can also be used to screen contrived libraries of microbes after co-culture in gel microspheres. For example, the technology as disclosed herein can have at least the following applications: (1) Phenotypic characterization of recovered clones: The recovered environmental colonies can be subjected to a variety of live-cell phenotypic screens to thoroughly characterize them. A few examples of phenotypic assays include, but are not limited to, antimicrobial susceptibility tests, cytotoxicity assays, and screening of the ability of a microorganism to adhere to mammalian cells. (2) Metagenomic studies: The proposed technology can facilitate rapid identification and characterization of previously unknown or unculturable species of bacteria from the environment by individually sequencing the recovered colonies. Knowledge of these new strain could help broaden the knowledge of the composition and functioning of complex microbiomes in the environment as well as inside the human body. This understanding could help humankind preserve natural environments via microbial bioremediation. (3) Epigenetic studies: Different strains of bacteria from diverse environments such as soil, freshwater, seawater, or human/animal gut microbiome could be subjected to co-culture with various other strains of microbes to determine changes in their transcriptome brought about by perturbations to their co-culture environment. (4) Bioproduction of novel active compounds: The proposed technology can help recover some previously unknown or uncharacterized microbes from the environment. Knowledge of the unique functions and life cycles of these organisms could lead to the discovery of novel active compounds that are of high interest in the fields of medicine and biotechnology.

In the following example, agarose gel microspheres and agarose-degrading enzyme agarase are used to further explain the systems and methods of the present disclosure as well demonstrate their successfulness.

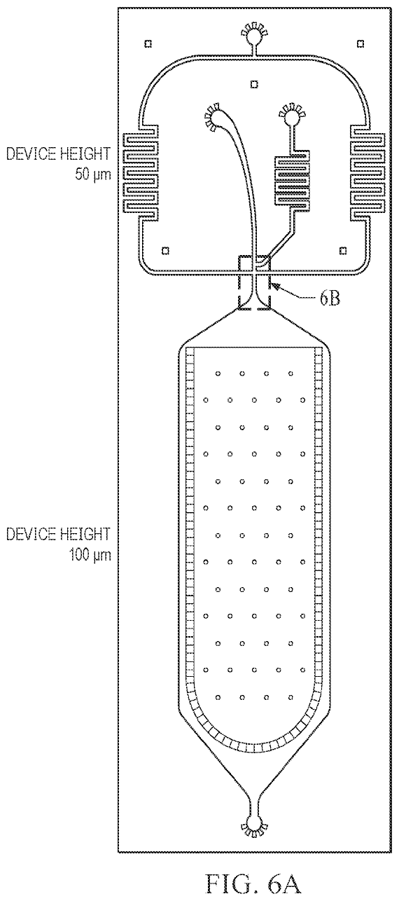

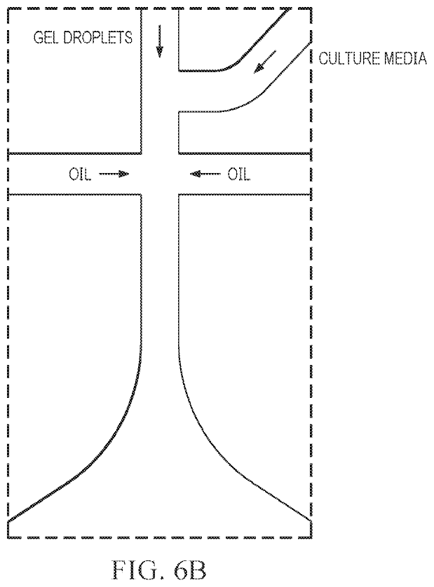

Agarose microsphere re-encapsulation performance. The device design and operation parameters shown here were optimized to maximize the single-gel microsphere re-encapsulation fraction in order to minimize cross-contamination of bacterial populations after release (FIGS. 6A-6B). To encapsulate the agarose microspheres with 60 μm diameter size, the width of the “droplet packing” channel at the inlet punch is 10× larger than the diameter of a single gel microsphere. The width gradually decreases to become slightly smaller than the diameter of a single gel microsphere, allowing the gel microspheres to be packed into a single line at high throughput without disrupting the flow within the microfluidic channel. This ensures that the gel microspheres consistently enter the droplet generator at a regular interval, thus enabling highly efficient re-encapsulation of single gel microspheres. It is important that the gel microspheres are slightly larger than the width of the channel before re-encapsulation to maximize the efficiency. Individual agarose gel microspheresbeing encapsulated into water-in-oil emulsion droplets. “Packed” gel microspheres are spaced out using culture media containing 10% (v/v) agarase enzyme, then encapsulated into separate water-in-oil droplets. It was also found that a single-spacing channel that intersects the packing channel orthogonally works best. Image of individual agarose gel microspheres being encapsulated into water-in-oil emulsion droplets were evaluated. “Packed” gel microspheres are spaced out using TSB culture media containing 10% (v/v) agarase enzyme, then encapsulated into separate water-in-oil droplets. With these design considerations, 75.2% single microsphere encapsulation efficiency at a total throughput of 3.2×10⁵ droplets/hour were obtained (total generation throughput was 4.3×10⁵).

FIGS. 6A-6B illustrate a schematic of a gel droplet to water-in-oil emulsion droplet transfer device. FIG. 6A illustrates the schematic of the gel droplet to water-in-oil emulsion droplet transfer device. FIG. 6B illustrates a zoomed in view of a junction point of the gel droplets, oil, and culture media. The height of the gel droplet re-encapsulation part of this device (top half of device) is adjusted to be slightly smaller than the gel droplets themselves.

This ensures efficient packing of the gel droplets. The imaging chamber (bottom half of device) is 100 μm in height. This ensures that the water-in-oil emulsion droplets are not squeezed during imaging. Images of individual agarose gel droplets being encapsulated into water-in-oil emulsion droplets were taken and analyzed. “Packed” gel droplets were spaced out using TSB culture media containing 10% (v/v) agarase enzyme, then encapsulated into separate water-in-oil droplets.

Degradation of agarose microspheres by agarase. Release of the microbial cargo encapsulated within the gel microspheres was enabled by an agarase enzyme (GH86) that hydrolyses the agarose backbone. Agarose has a linear backbone with repeating disaccharide subunits of 3-O-β-D-galactose (GAL) and 4-O-α-3,6-anhydro-L-galactose (AHG). Agarolysis can be performed by a family of agarase glycoside hydrolase (GH) enzymes that liberate the GAL and AHG. GH86 is an endo-acting agarase that hydrolyses the β-1,4 linkages of the agarose backbone. Agarase was expressed and purified based on the GH86.

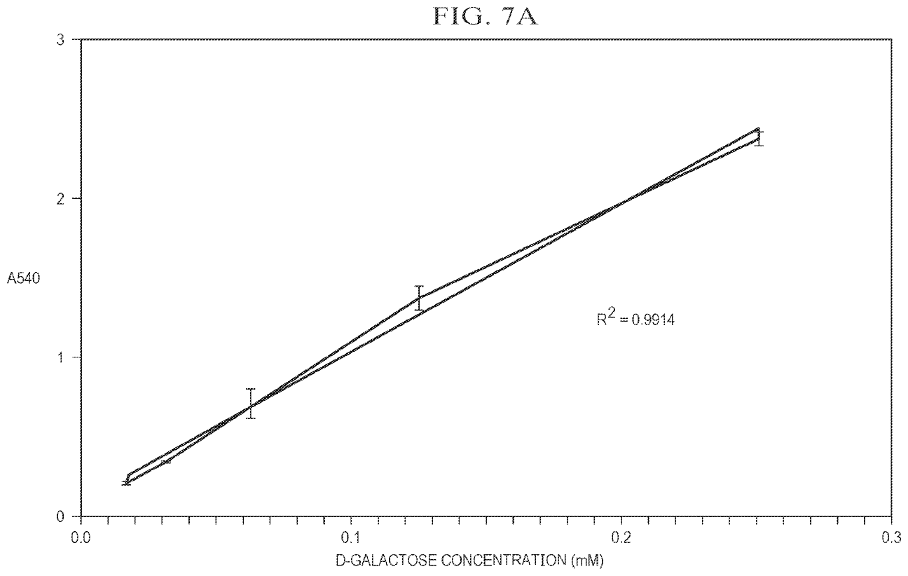

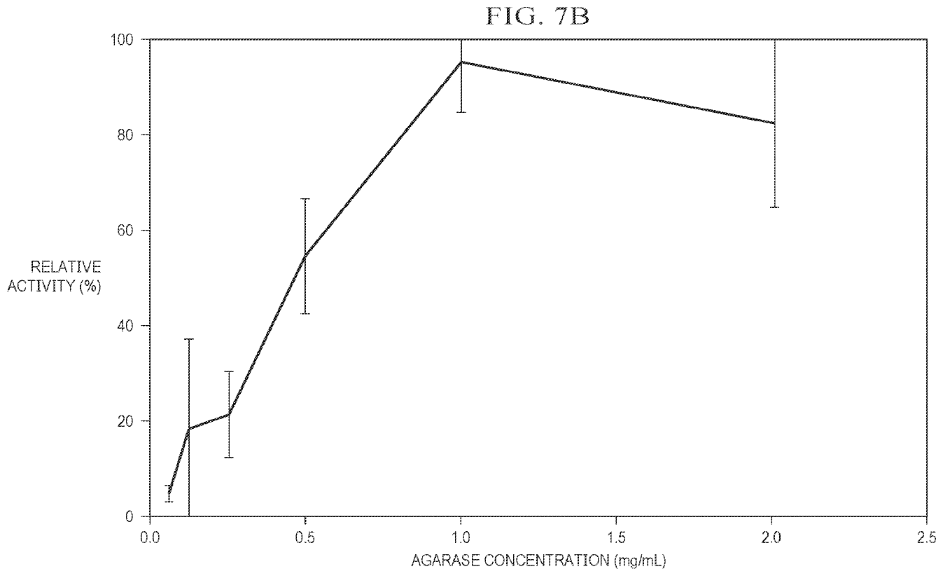

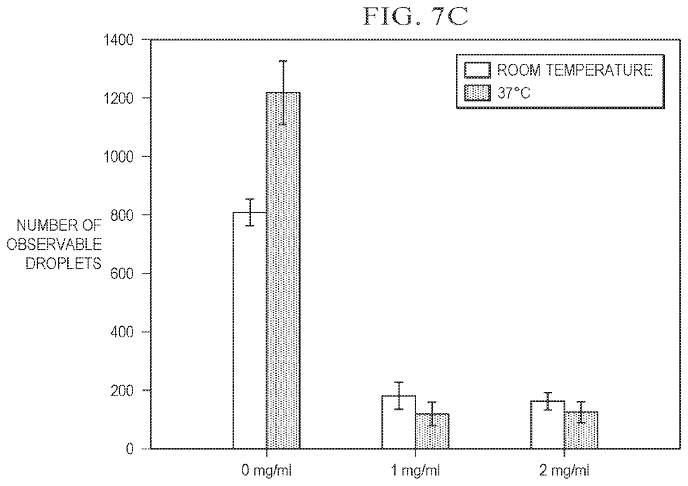

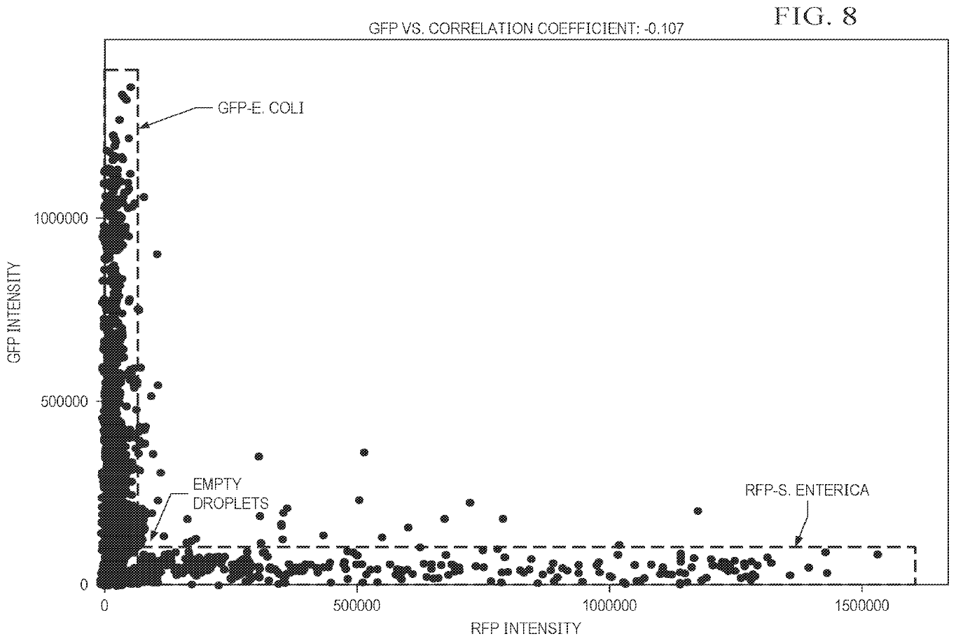

Once purified, the agarolytic activity of the purified enzyme was measured using a 3, 5-dinitrosalicyclic acid (DNS)-based colorimetric assay. First, a standard curve was prepared by incubating the DNS reagents with concentrations of galactose starting at 1 μM and up to 250 nM. The reduction of DNS by sugars like galactose produces color hence increasing the absorbance of the solution. Absorbance was measured at 540 nm. A strong positive correlation was observed between the concentration of galactose and absorbance measurements (R²=0.9914; FIG. 7A).

Next, a solution containing 1 mg/mL agarose (A5030 ultra low temperature melting) in 20 mM Tris HCl buffer was incubated with various concentrations of the purified agarase GH86 at 37° C. overnight before performing the DNS assay. The absorbance (at 540 nm) of this mixture was measured using a plate reader and the relative activity was calculated. Highest relative activity was observed at a concentration of 1 mg/mL agarase (FIG. 7B).

Finally, a visual confirmation of agarose microbeads being degraded by agarase was performed. 1% and 2% agarose solutions were emulsified using a single flow focusing droplet generator. The emulsion was allowed to solidify at 4° C. for 15 minutes and was then washed and resuspended in phosphate buffered saline (PBS). 2 mg/mL agarase was added at a two-fold dilution to this suspension and the morphology of the droplets was observed over time at various temperatures. It was determined that a 2 mg/ml solution of agarose can fully degrade 60 μm (diameter) agarose microdroplets within 5 minutes at 37° C. (FIG. 7C).

FIG. 7 illustrates characterization of purified agarase GH86 activities. FIG. 7A shows a standard curve of DNS activity assay. The DNS reagents were incubated with galactose standards at different concentrations (1 μM to 250 nM), and absorbance measured at 540 nm. The absorbance of the sample is seen to increase significantly with the higher concentration standards, indicating a positive correlation between the assay readout and input galactose concentration. FIG. 7B shows relative activity of the purified agarase enzyme. Various concentrations of agarase were incubated with 1 mg/mL agarose and the relative activity was measured using the DNS assay. Error bars are standard deviation (N=3). The relative activity of agarase can be seen to increase significantly until a concentration of 1 mg/mL agarase. Beyond that, the data suggests that the relative activity is constant. FIG. 7C shows analysis of images of the degradation of microdroplets made of 1% and 2% agarose using agarase. The number of observable agarose droplets reduces significantly in the presence of agarase. The samples treated with 1 mg/mL and 2 mg/mL agarase had similar number of observable droplets. This observation is consistent with the results of the previous graph which suggests that the highest activity of agarose occurs at 1 mg/mL. Fewer droplets are observed at 37° C. than at room temperature (RT) indicating an increased enzyme activity at cell culture temperatures. Images of the degradation of microdroplets made of 1% and 2% agarose using agarose were analyzed. Droplets can be seen to lose their definition in the images of samples containing some agarase. Fewer droplets can be seen in images of samples incubated at 37° C. Based on the analysis of these images, 2 mg/mL agarase was determined to work best at a temperature of 37° C.

Release of co-cultured bacteria from agarose microspheres. The re-encapsulated droplets were incubated in a basket-like droplet trapping chamber at 37° C. for 2.5 hours. Within a few minutes after re-encapsulation, the agarase had hydrolyzed the gel and released the microbial “carbo” into the “parent” water-in-oil emulsion droplets. Over time, the RFP or GFP intensities of the “parent” droplets increased as the bacteria multiplied, indicating that viable bacteria had been recovered. A low correlation of −0.107 between the RFP and GFP intensities of the “parent” droplets after 2.5 hours of culture indicate a high degree of separation between the two strains of bacteria (FIG. 8).

Images of single GFP-E. coli and RFP-S. enterica cells encapsulated in 60 μm agarose microspheres were taken. Over several hours of culture in TSB media, the single cells are held in place by the agarose and form colonies. Images were taken of GFP-E. coli and RFP-S. enterica-containing agarose microspheres re-encapsulated within water-in-oil droplets. Zoomed-in images showing an agarose microsphere containing a single GFP-E. coli colony being degraded by agarase enzyme after re-encapsulation were taken. The released bacteria are no longer confined by the gel matrix and are free to occupy the entire volume of the water-in-oil emulsion droplet.

Results of re-encapsulating cell-containing gel droplets into water-in-oil emulsion droplets using eBRIDGE. Images showing single GFP-E. coli and RFP-S. enterica cells encapsulated in 60 μm agarose microdroplets were analyzed. Over several hours of culture in TSB media, the single cells are held in place physically isolated from one another by the agarose, and form colonies. During this process, diffusion-mediated inter-cell communication pathways (e.g., exchange of metabolites, OMVs, etc.), that might be critical for the survival of certain rare/previously unculturable species of environmental bacteria, are maintained. Single-cell-derived colonies can be seen forming within some of the gel droplets, and no gel droplet contains both green and red colonies. Images of GFP-E. coli and RFP-S. enterica-containing agarose droplets re-encapsulated within water-in-oil droplets were also analyzed. These re-capsulated droplets were cultured at 37° C. for 2.5 h to observe the growth and release of the bacteria from the agarose droplets. The intensity of both fluorescence channels seems to increase over time, indicating healthy growth of both strains.

FIG. 8 illustrates analysis of the contents of the water-in-oil droplets. Fluorescence

analysis of ˜1800 droplets shows very low correlation between the GFP and RFP intensities of the water-in-oil droplets. A scatter plot of the GFP and RFP intensities shows an L-shaped distribution of two independent variables, suggesting a good degree of separation between the GFP and RFP microbes. The correlation coefficient between GFP and RFP intensities of the analyzed droplets is −0.107. Zoomed-in images showing an agarose droplet containing a single GFP-E. coli colony being degraded by agarase enzyme after re-encapsulation were additionally analyzed. It was shown that the released bacteria are no longer confined by the gel matrix and are free to occupy the entire volume of the water-in-oil droplet. This is in stark contrast to “before degradation”, when the entire colony occupies a small volume roughly at the center of the droplet.

Re-encapsulated droplets containing S. enterica was identified as 285, re-encapsulated droplets containing E. coli was identified as 707, re-encapsulated droplets containing S. enterica and E. coli was identified as 43, and empty droplets was identified as 1762.

Although the description here is for the case of agarose and agarose-degrading enzyme agarase, other gel and degradation reagent combinations can be also utilized. For example, the use of temperature-sensitive gel can be utilized (e.g., MEBIOL® gel), where the change in temperature results in gel microspheres to change into liquid phase, thus resulting in release of the gel microsphere's cargo into the water-in-oil emulsion droplet.

In view of the above, in an embodiment, the present disclosure pertains to a process (eBRIDGE) to encapsulate either individual microbial cells from a single or multi-organism suspension, or individual mammalian cells from a genetically heterogenous population, or individual mammalian cells from a genetically identical population into gel microspheres, cultivate to produce viable single-cell-derived colonies of the encapsulated organisms, transition each single-cell derived colony into separate water-in-oil emulsion droplets, followed by releasing the cellular cargo from the gel microspheres using enzyme-based degradation of the gel material. In some embodiments, the gel droplet encapsulation chip can be one of a variety of microfluidic emulsion generators including, but not limited to, co-flowing, flow focusing, and cross-flowing designs.

In some embodiments, one of many gel materials can be used, including, but not limited, to agarose (and other structurally distinct polysaccharides), polyethylene glycol, various compositions including polyethylene glycol 6k, polyethylene glycol-diacrylate, and the like, gelatin methacryloyl, MEBIOL®, and combinations of the same and like. In some embodiments, encapsulation entails the capturing of single microbial or mammalian cells into a microsphere made of a gel material is utilized for encapsulation.

In some embodiments, gel microspheres are initially produced as a two-phase emulsion droplet, but eventually re-suspended in an aqueous medium (water, PBS, culture media, and the like) after a carrier oil washing step. In some embodiments, the encapsulated cells can then be transferred to any on-or off-chip chamber and cultured in any desired culture medium, depending on the type of biological sample used. In some embodiments, the culture chamber can be any design that allows all the gel microspheres to share a common culture medium.

In some embodiments, this can include designs that allow free-flow of fresh culture medium over/through the gel microspheres, or static containers, including, but not limited to, well-plates, culture tubes, centrifuge tubes, and combinations of the same and like. In some embodiments, the culture chambers can be incubated at any desired temperature and atmospheric condition (e.g., anaerobic, 5% CO₂, etc.).

In some embodiments, the cultured microspheres can then be washed and compacted. It should be noted that, in some embodiments, this washing can be used to remove escaped cells, or cells attached to the exterior of the microsphere. In some embodiments, compacting the gel microspheres entails removing most excess aqueous solution that the microspheres were suspended in. In some embodiments, this can be achieved by transferring the microspheres to a syringe before centrifuging them using a specially designed holder, then aspirating any supernatant. In some embodiments, features of the syringe holder include: (1) various stops to lock the plunger into place and add as much sample as necessary to the syringe; (2) compatibility with different swing out centrifuge rotors; and (3) structural integrity up to at least 3000 G centrifugal force. In some embodiments, a microfluidic gel droplet compacting chip can also be used to achieve this.

In some embodiments, the compacted microspheres are then packed. In some embodiments, gel droplet packing entails organizing the compacted gel microspheres into a single file for feeding into the re-encapsulation device using a gradually tapered microfluidic channel. In some embodiments, the initial channel width of the gel droplet packing region can be arbitrarily large, but the final width can be slightly (5-7 μm) smaller than the gel droplet diameter.

In some embodiments, the packed gel microspheres are then spaced out. In some embodiments, the spacing region introduces an aqueous stream at an angle (any angle larger or smaller than 90°) and separates adjacent gel microspheres for encapsulation into separate water-in-oil droplets. In some embodiments, the spacing fluid can contain fresh culture media along with an enzyme. In some embodiments, this can be one of several depending on the gel material used, including, but is not limited, to agarase GH86, collagenase, and combinations of the same and like to enable release of cellular cargo from within the gel microspheres as the enzyme degrades the gel material.

In some embodiments, the enzyme can also be added later via one of many droplet microfluidic manipulation techniques, including, but not limited to, droplet pairing/merging, pico-injection, and the like. In some embodiments, the spacing stream can also include any reagents needed for downstream characterization assays, including, but not limited to, antibiotics, fluorescence dyes, colorimetric dyes, beads, and combinations of the same and like. In some embodiments, the spaced microspheres are then re-encapsulated in water-in-oil droplets. In some embodiments, this re-encapsulation can be achieved using any microfluidic emulsion generator design including, but not limited to co-flowing, flow focusing and cross-flow designs. In some embodiments, the re-encapsulated microspheres can be manipulated using any droplet microfluidics techniques. In some embodiments, the resulting droplets can be transferred to any on-or off-chip chamber to be cultured at any desired temperature and atmospheric condition.

In some embodiments, the resulting droplets can be used as an input for another droplet microfluidics system, including, but not limited to, picoinjection devices, fluorescence assisted droplet sorting devices, droplet-based phenotype interrogation systems, and combinations of the same and like. In some embodiments, the resulting droplets can be detected using a variety of modalities, including, but not limited to, image-based detection, impedance-based detection, and combinations of the same and like. In the case of imaging, in some embodiments, the resulting droplets can be imaged using an on-or off-chip imaging chamber. In some embodiments, the microfluidic functionalities described above can be integrated onto a single chip or can be separate chips.

In an additional embodiment, the present disclosure pertains to a process (eBRIDGE) to encapsulate either individual microbial cells from a single or multi-organism suspension, or individual mammalian cells from a genetically heterogenous population, or individual mammalian cells from a genetically identical population into gel microspheres, cultivate to produce viable single-cell derived colonies within the encapsulated microorganisms, transition each single-cell derived colony into separate water-in-oil emulsion droplets, followed by releasing the cellular cargo from the gel microspheres using a temperature-dependent dissolution/degradation of the gel material.

In some embodiments, the gel droplet encapsulation chip can be one of a variety of

microfluidic emulsion generators, including, but not limited, to co-flowing, flow focusing and cross-flowing designs. In some embodiments, one of many gel materials can be used, including, but not limited, to MEBIOL® and the like. In some embodiments, encapsulation entails the capturing of single bacterial cells into a microsphere made of a gel material. In some embodiments, gel microspheres are initially produced as a two-phase emulsion droplet, but eventually re-suspended in an aqueous medium, including, for example, water, PBS, culture media, and the like after washing off the carrier oil. In some embodiments, the encapsulated cells can then be transferred to any on-or off-chip chamber and cultured in any desired culture medium depending on the type of biological sample used.

In some embodiments, the culture chamber can be any design that allows all the gel microspheres to share a common culture medium. In some embodiments, this can include designs that allow free-flow of fresh culture medium over/through the gel microspheres, or static containers, including, but not limited to, well-plates, culture tubes, centrifuge tubes, and combinations of the same and like. In some embodiments, the culture chambers can be incubated at any desired temperature and atmospheric condition (e.g., anaerobic, 5% CO₂, etc.).

In some embodiments, the cultured microspheres can then be washed to get rid of any escaped cells and compacted. In some embodiments, compacting the gel microspheres entails removing any excess aqueous solution that the microspheres were suspended in. In some embodiments, this can be achieved by transferring the microspheres to a syringe before centrifuging them using a specially designed holder, then aspirating any supernatant. In some embodiments, a microfluidic gel droplet compacting chip can also be used to achieve this.

In some embodiments, the compacted microspheres are then packed. In some embodiments, gel droplet packing entails organizing the compacted gel microspheres into a single file for feeding into the re-encapsulation device using a gradually tapering microfluidic channel. In some embodiments, the initial channel width of the gel droplet packing region can be arbitrarily large, but the final width can be slightly (˜5 μm) smaller than the gel droplet diameter.

In some embodiments, the packed gel microspheres are then spaced out. In some embodiments, the spacing region introduces an aqueous stream at an angle (any angle larger or smaller than 90°) and separates adjacent gel microspheres for encapsulation into separate water-in-oil droplets. In some embodiments, the spacing fluid can contain anything from fresh culture media to any reagents needed for downstream characterization assays, including, but not limited to, antibiotics, fluorescence dyes, colorimetric dyes, beads, and combinations of the same and like.

In some embodiments, the spaced microspheres are then re-encapsulated in water-in-oil droplets. In some embodiments, this can be achieved using any microfluidic emulsion generation design, including, but not limited to, co-flowing, flow focusing and cross-flow designs. In some embodiments, the temperature of the re-encapsulated gel microspheres can be manipulated using an underlying channel carrying heated/cooled water to trigger a temperature-dependent dissolution of the gel material and release the cellular cargo. In some embodiments, this step can also be done by collecting the re-encapsulated droplets off-chip and incubating them at the required temperature.

In some embodiments, the re-encapsulated microspheres can be manipulated using any conventional droplet microfluidics techniques. In some embodiments, the resulting droplets can be transferred to any on-or off-chip chamber to be cultured at any desired temperature and atmospheric condition. In some embodiments, the resulting droplets can be used as an input for another droplet microfluidics system, including, but not limited to, picoinjection devices, fluorescence assisted droplet sorting devices, droplet-based phenotype interrogation systems, and combinations of the same and like. In some embodiments, the resulting droplets can be imaged using an-on-or off-chip imaging chamber. In some embodiments, the microfluidic functionalities can be integrated onto a single chip or separate chips.

In an additional embodiment, the present disclosure pertains to a process to create co-cultures of various distinct microbes within a diverse community, or genetically distinct mammalian cells within a population of genetically distinct mammalian cells. In some embodiments, single cells from a diverse microbial community or mammalian cell population can be encapsulated into gel microspheres using any one or more of the techniques described in Implementations #1-4 above. In some embodiments, the microspheres can then be resuspended in aqueous culture medium and compacted and packed as described in any one or more of Implementations #1-4 above. In some embodiments, multiple gel microspheres can then be re-encapsulated into a single water-in-oil droplet to enable co-culture of multiple different strains from within the original community. In some embodiments, the growth of cells within the resulting emulsion can be observed and specific droplets of interest can be recovered using conventional droplet microfluidic detection and sorting techniques.

In a further embodiment, the present disclosure pertains to a process to pair microspheres made of different gel materials with cells from a single-organism microbial culture or population of genetically identical mammalian cells to assay a target organism's ability to interact with, adhere to, consume or degrade various gel materials. In some embodiments, microspheres of different gel materials can be generated separately using any microfluidic emulsion generators. In some embodiments, the types of generators that can be used can include, but are not limited to, co-flow, flow focusing and cross-flow droplet generators. In some embodiments, the gel microspheres can be re-suspended in an aqueous medium and mixed on- or off-chip. In some embodiments, using any one or more of the processes in Implementations #1-4, discussed above, the microspheres can be compacted and packed. In some embodiments, the microspheres can then be spaced out using a concentrated suspension of clonal microbial or mammalian cells. In some embodiments, this suspension can be made up of any culturable microbial or mammalian species. In some embodiments, the spaced microspheres can then be re-encapsulated in water-in-oil droplets along with the cells using any microfluidic droplet generator including, but not limited, to co-flow, flow focusing and cross-flow designs. In some embodiments, the resulting water-in-oil droplets can be incubated on-or off-chip at any desired temperature or atmospheric condition. In some embodiments, the droplets can be detected using a wide variety of techniques including, but not limited, to fluorescence or impedance-based detection methods. In a particular example, the droplets can be observed using conventional microscopy techniques or interrogated and sorted using any droplet microfluidic techniques. In some embodiments, the final process achieves 98-100% separation of different gel materials with less than 20-25% of the resulting water-in-oil droplets being empty (containing no gel microspheres).

In a further embodiment, the present disclosure pertains to a process to pair single cells from multi-organism mixtures, or a population of genetically identical cells (microbial or mammalian), with microspheres made of a specific gel material to identify and recover specific species or strains based on their ability to interact with, adhere to, consume or degrade the gel material. In some embodiments, empty microspheres made of a specific gel material including, but not limited, to polysaccharides (e.g., agarose and the like), polymers (e.g., PEG, gelatin methacryloyl, MEBIOL®, and the like) can be generated using any microfluidic emulsion generator device. In some embodiments, the gel microspheres can be resuspended in an aqueous medium. In some embodiments, using any one or more of the processes in Implementations #1-4, discussed above, these gel microspheres can be compacted and packed.

In some embodiments, the packed gel microspheres can then be spaced using a diluted multi-organism microbial or mammalian cell culture. In some embodiments, the specific dilution ratio can be adjusted based on the desired water-in-oil droplet size to enable co-encapsulation of single cells along with single gel microspheres. In some embodiments, the resulting water-in-oil droplets can be incubated on-or off-chip at any desired temperature or atmospheric condition. In some embodiments, the droplets can be detected using a wide variety of techniques, including, but not limited, to fluorescence or impedance-based detection methods. In a particular example, the droplets can be observed using conventional microscopy techniques or interrogated and sorted using any droplet microfluidic techniques.

Although various embodiments of the present disclosure have been illustrated in the

accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A method to encapsulate at least one of individual microbial cells from a single-or multi-organism suspension, individual mammalian cells from a genetically heterogenous population, or individual mammalian cells from a genetically identical population into gel microspheres, the method comprising:
encapsulating single-cells into the gel microspheres using a gel material;
cultivating the gel microspheres to produce viable single-cell-derived colonies of encapsulated organisms;
transitioning each single-cell derived colony into separate water-in-oil emulsion droplets; and
releasing cellular cargo from the gel microspheres using enzyme-based degradation of the gel material used for encapsulation into the water-in-oil emulsion droplets.

2. The method of claim 1, wherein the encapsulating utilizes a microfluidic emulsion generator selected from the group consisting of co-flowing, flow focusing, cross-flowing designs, and combinations thereof.

3. (canceled)

4. The method of claim 1, wherein the encapsulating comprises capturing of single microbial or mammalian cells into a gel microsphere made of the gel material.

5-6. (canceled)

7. The method of claim 1, wherein the encapsulated single-cells are transferred to an on- or off-chip chamber and cultured in a culture medium depending, at least in part, on a type of biological sample used.

8-12. (canceled)

13. The method of claim 1, further comprising washing and compacting cultured gel microspheres.

14. The method of claim 13, wherein the washing removes escaped cells or cells attached to an exterior of the cultured gel microsphere.

15. The method of claim 13, wherein the compacting the cultured gel microspheres comprises removing most excess aqueous solution that the cultured gel microspheres were suspended in so that they are next to each other with minimum spacing in between the cultured gel microspheres.

16. The method of claim 13, comprising transferring the cultured gel microspheres to a syringe before centrifuging them using a specially designed holder and then aspirating any supernatant.

17. The method of claim 16, wherein the syringe holder includes at least one of various stops to lock the plunger into place and add as much sample as necessary to the syringe, is compatible with different swing out centrifuge rotors, or structural integrity up to at least 3000 G of centrifugal force.

18. The method of claim 1, wherein a microfluidic gel microsphere compacting chip is used such that cultured gel microspheres are compacted.

19. The method of claim 1, further comprising packing the gel microspheres.

20. The method of claim 19, wherein the packing comprises organizing the gel microspheres into a single file for flowing them into the re-encapsulation device using a gradually tapered microfluidic channel.

21. (canceled)

22. The method of claim 1, further comprising spacing out the gel microspheres.

23-27. (canceled)

28. The method of claim 1, further comprising re-encapsulating spaced gel microspheres in water-in-oil emulsion droplets.

29. The method of claim 28, wherein the re-encapsulating is achieved by using a microfluidic droplet emulsion generator design selected from the group consisting of a co-flow design, a flow focusing design, a cross-flow design, and combinations thereof.

30. (canceled)

31. The method of claim 30, wherein resulting droplets are transferred to an on-or off-chip chamber to be cultured at any desired temperature and atmospheric condition.

32. The method of claim 30, wherein resulting droplets are used as an input for another droplet microfluidics system selected from the group consisting of pico-injection devices, fluorescence assisted droplet sorting devices, droplet-based phenotype interrogation systems, and combinations thereof.

33. The method of claim 30, wherein resulting droplets are detected using a modality selected from the group consisting of image-based detection, fluorescence-based detection, luminescence-based detection, spectroscopy-based detection, impedance-based detection, and combinations thereof.

34. The method of claim 30, wherein resulting droplets are detected using image-based detection via an on-or off-chip imaging chamber.

35. The method of claim 1, wherein microfluidic functionalities are integrated onto a single chip or separate chips.
